(19) United States
(12) Patent Application Publication
(10) Pub. No.: US 2024/0090941 A1
(43) Pub. Date: Mar. 21, 2024

(54) PROBE SYSTEMS AND METHODS FOR IRREVERSIBLE ELECTROPORATION

(71) Applicant: Varian Medical Systems, Inc., Palo Alto, CA (US)

(72) Inventors: Oliver Hornung, Unterleinleiter (DE); Jens-Christoph Georgi, Oberasbach (DE); Alois Regensberger, Poxdorf (DE); Peter Fischer, Gerhardshofen (DE)

(21) Appl. No.: 17/933,266

(22) Filed: Sep. 19, 2022

(51) Int. Cl. A61B 18/14

(52) U.S. Cl. CPC A61B 18/1492; A61B 2017/00991

(57) ABSTRACT

An ablation system includes a retractable sheath including a lumen; a first electrode in the lumen; a pre-bent telescopic tube extendable from the lumen and including a second electrode, wherein the first electrode and the second electrode are configured to deliver an electric field energy to target tissue in a patient.

### TECHNICAL FIELD

The disclosure relates to irreversible electroporation probes. More specifically, the disclosure relates to irreversible electroporation probe systems that include two electrodes and methods of performing irreversible electroporation therapy using the same.

### BACKGROUND

Irreversible electroporation (IRE) is a non-thermal ablative therapy technique that uses high-voltage, low-energy DC current pulses to induce cell death. Thermal ablative technologies such as radiofrequency ablation, microwave ablation, and cryoablation have several applications in oncology but have limitations. IRE overcomes some of these limitations.

IRE is a tissue ablation technique in which micro to millisecond electrical pulses are delivered to undesirable tissue to produce cell necrosis through irreversible cell membrane permeabilization. IRE affects only the cell membrane and no other structure in the tissue.

Ablation procedures based on the IRE principle use very strong, ultra-short electrical fields to destroy the cell membrane of target cells (e.g., tumors or cancerous tissue). During treatments, an electrical field is applied between at least two needle-shaped probes including electrodes that must be placed in parallel about the target region. Although at least two probes are required to create a treatment zone, additional probes can be used depending on the size of the lesion or target region. Additional probes can be placed in parallel with each other about different locations of a 3D target region. In which case, the electrical energy is delivered between two probes at a time. IRE has been successful in treating liver, prostate, and pancreatic cancer and is being investigated for other treatments.

Prior to ablation, correct placement of the electrode probes is critical for a successful ablation result. Conventionally, IRE is performed using image-guidance (i.e., with computed tomography (CT) or ultrasound imaging) under general anesthesia. Probe placement may have to be repeated several times until the correct positioning is achieved. Following placement of the probes with imaging guidance, a three-dimensional (3D) CT scan image can be obtained to confirm the position of the probes and the distance between each pair. Correctly positioning the IRE probes is time consuming and expensive. Thus, there exists a need for improved IRE probes that are less invasive and can be positioned more accurately and more quickly.

### SUMMARY OF THE DISCLOSURE

In various embodiments of the present disclosure, systems and methods are provided that are less invasive than conventional IRE treatments that require use of more probes. IRE ablation probe systems of the present disclosure can include at least two electrodes in one device used in IRE therapy, eliminating the need for two separate probes each with one electrode.

IRE ablation probe systems of the present disclosure can also include an imaging sensor. Therefore, systems and methods of the present disclosure can obtain imaging information that describe the progress of an IRE ablation treatment during the procedure. This information is an improvement over existing and traditional methods of treatment by providing real-time feedback regarding the ablation of a target tissue during treatment. This information can improve the effectiveness of the treatment and can reduce the likelihood that subsequent treatments are performed. The systems and methods of the present disclosure can assist a medical professional to determine whether all or a desired portion of the target tissue has been destroyed during treatment. Furthermore, the systems and methods of the present disclosure can reduce and/or limit harm to healthy tissues that may be located close to the target tissue.

In some examples, ultrasound imaging can be integrated into IRE probe systems. In some examples, all-optical imaging can be integrated into IRE probe systems. Imaging sensors in IRE probe systems can provide imaging data of the ablation volume that is not possible with existing handheld or external imaging probes.

Also, the embodiments of the present disclosure can be embodied as a method, of which an example has been provided. The acts performed as part of the method can be ordered in any suitable way. Accordingly, embodiments can be constructed in which acts are performed in an order different than illustrated, which can include performing some acts concurrently, even though shown as sequential acts in illustrative embodiments.

In an embodiment of the present disclosure, an ablation system can include a retractable sheath including a lumen; a first electrode in the lumen; a bendable telescopic tube extendable from the lumen and including a second electrode, wherein the first electrode and the second electrode are configured to deliver an electric field energy to target tissue in a patient.

In one aspect, the first electrode is exposed to the outside when the retractable sheath is retracted.

In another aspect, the bendable telescopic tube is pre-bent with two opposing 90-degree bends.

In another aspect, the bendable telescopic tube is pre-bent with a 180-degree bend.

In another aspect, the second electrode is extendable from the bendable telescopic tube.

In another aspect, when the bendable telescopic tube is extended from the lumen and the second electrode is extended from the bendable telescopic tube, the first electrode and the second electrode are arranged parallel to each other.

In an embodiment, the ablation system can further include an imaging sensor positioned to obtain imaging data of the target tissue. In an aspect, the imaging sensor is configured to operate simultaneously with operation of the first and second electrodes.

In another embodiment of the present disclosure, an ablation system includes a retractable sheath including a lumen; a first bendable telescopic tube extendable from the lumen and including a first electrode; and a second bendable telescopic tube extendable from the lumen and including a second electrode, wherein the first electrode and the second electrode are configured to deliver an electric field energy to target tissue in a patient.

In an aspect, the first bendable telescopic tube and the second bendable telescopic tube are each pre-bent with a 90-degree bend.

In another aspect, the first electrode is extendable from the first bendable telescopic tube; and the second electrode is extendable from the second bendable telescopic tube.

In another aspect, when the first bendable telescopic tube is extended from the lumen, the first electrode is extended from the first bendable telescopic tube, the second bendable telescopic tube is extended from the lumen, and the second electrode is extended from the second bendable telescopic tube, the first electrode and the second electrode are arranged parallel to each other.

In another embodiment of the present disclosure, a method of performing irreversible electroporation (IRE) therapy includes retracting a sheath of an IRE probe system that is located at or near target tissue in a patient; extending a first bendable telescopic tube from a lumen of the IRE probe system; extending a first electrode from the first bendable telescopic tube; and energizing an electrical field between the first electrode and a second electrode of the IRE probe system that is parallel to the first electrode to ablate the target tissue in a first orientation.

In an aspect, the method can further include retracting the first electrode; retracting the first bendable telescopic tube; extending the sheath; rotating the IRE probe system; re-extending the first bendable telescopic tube from the lumen of the IRE probe system; re-extending the first electrode from the first bendable telescopic tube; and energizing an electrical field between the first electrode and the second electrode to ablate the target tissue in a second orientation.

In another aspect, the method can further include extending a second bendable telescopic tube from the lumen of the IRE probe system; and extending the second electrode from the second bendable telescopic tube.

In another aspect, the method can further include retracting the first electrode and the second electrode; retracting the first bendable telescopic tube and the second bendable telescopic tube; extending the sheath; rotating the IRE probe system; re-extending the first bendable telescopic tube from the lumen of the IRE probe system; re-extending the first electrode from the first bendable telescopic tube; re-extending the second bendable telescopic tube from the lumen of the IRE probe system; re-extending the second electrode from the second bendable telescopic tube; and energizing an electrical field between the first electrode and the second electrode to ablate the target tissue in a second orientation.

In another aspect, the method can further include obtaining imaging data from an imaging sensor of the IRE probe system.

In another aspect, the method can further include simultaneously energizing an electrical field between the first electrode and the second electrode and obtaining imaging data from the imaging sensor.

The above and other features, elements, characteristics, steps, and advantages of the present invention will become more apparent from the following detailed description of preferred embodiments of the present invention with reference to the attached drawings.

### BRIEF DESCRIPTION OF THE DRAWINGS

The features and advantages of the present disclosures will be more fully disclosed in, or rendered apparent by the following detailed descriptions of example embodiments. The detailed descriptions of the example embodiments are to be considered together with the accompanying drawings wherein like numbers refer to like parts and further wherein:

FIG. 1 shows an ablation system of the related art.

FIG. 2 is a side view of an example ablation probe system according to the present disclosure.

FIG. 3 is a side view of an ablation probe system of FIG. 2.

FIG. 4 is a side view of another example ablation probe system of FIG. 2.

FIG. 5 is a side view of another example ablation probe system according to the present disclosure.

FIG. 6 is a side view of an ablation probe system of FIG. 5.

FIG. 7 is a side view of another example ablation probe system according to the present disclosure.

FIG. 8 is a side view of another example ablation probe system according to the present disclosure.

FIG. 9 is a flowchart of an example method of performing an ablation treatment in accordance with the present disclosure.

### DETAILED DESCRIPTION

The description of the preferred embodiments is intended to be read in connection with the accompanying drawings, which are to be considered part of the entire written description of these disclosures. While the present disclosure is susceptible to various modifications and alternative forms, specific embodiments are shown by way of example in the drawings and will be described in detail herein. The objectives and advantages of the claimed subject matter will become more apparent from the following detailed description of these exemplary embodiments in connection with the accompanying drawings.

It should be understood, however, that the present disclosure is not intended to be limited to the particular forms disclosed. Rather, the present disclosure covers all modifications, equivalents, and alternatives that fall within the spirit and scope of these exemplary embodiments. The terms “couple,” “coupled,” “operatively coupled,” “operatively connected,” and the like should be broadly understood to refer to connecting devices or components together either mechanically, electrically, wired, wirelessly, or otherwise, such that the connection allows the pertinent devices or components to operate (e.g., communicate) with each other as intended by virtue of that relationship.

In the present disclosure the singular forms “a,” “an,” and “the” include the plural reference, and reference to a particular numerical value includes at least that particular value, unless the context clearly indicates otherwise. When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” preferably refers to a value of 7.2 to 8.8, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, “2-5”, and the like. In addition, when a list of alternatives is positively provided, such listing can be interpreted to mean that any of the alternatives may be excluded, e.g., by a negative limitation in the claims. For example, when a range of “1 to 5” is recited, the recited range may be construed as including situations whereby any of 1, 2, 3, 4, or 5 are negatively excluded; thus, a recitation of “1 to 5” may be construed as “1 and 3-5, but not 2”, or simply “wherein 2 is not included.” It is intended that any component, element, attribute, or step that is positively recited herein may be explicitly excluded in the claims, whether such components, elements, attributes, or steps are listed as alternatives or whether they are recited in isolation.

The IRE probes of the present disclosure can reduce the number of probes required during an IRE procedure. Less probes are desirable because insertion of IRE probes into tissue is invasive. Less probes inserted into a patient means less chances for probes to cause bleeding, tissue damage, or other undesirable affects. Additionally, it takes time by a medical professional to properly align electrodes of IRE probes in parallel for treatment using imaging guidance. The IRE probes of the present invention can each include two electrodes that automatically position themselves to be in parallel with each other. This can speed the IRE procedure and require less time for the patient to be under anesthesia.

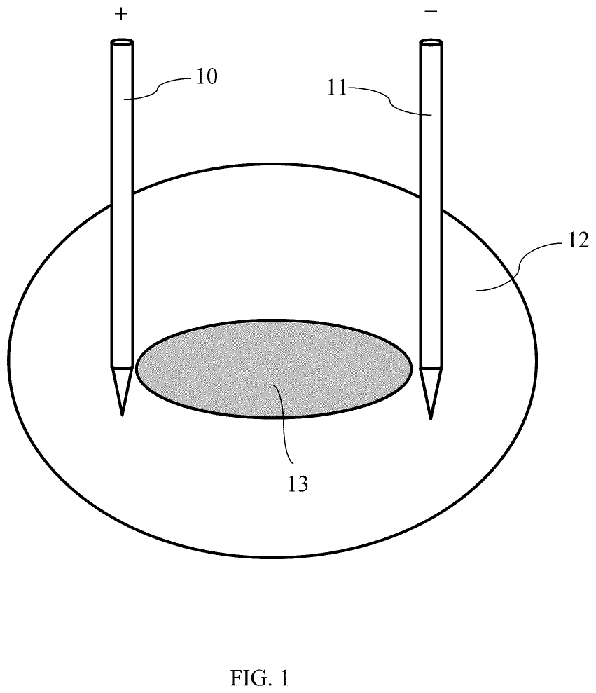

IRE probes with electrodes are often used during treatments to target undesirable tissues such as tumors or cancer cells. During such procedures, a first IRE probe can be inserted into a body and be positioned at or near the target tissue. A second IRE probe can be inserted into the body and be positioned on the other side of the target tissue such that an electrode of the second IRE probe is parallel to an electrode of the first IRE probe. This is illustrated in FIG. 1 that shows two needle-shaped probes with electrodes, positive electrode 10 and negative electrode 11, placed through tissue 12 to be parallel with each other about a target region 13. An electrical generator (not shown) delivers high-voltage, low-energy DC current through the probes 10 and 11 connected to the generator. Although at least two monopolar probes are required to create a treatment zone, additional probes can be used depending on the size of the lesion or target region. Additional probes can be placed in parallel with each other about different locations of a 3D target region. Therefore, by driving two electrodes of the IRE probes at any one time, electrical fields can be created at different orientations around the target tissue.

For insertion into tissue, an IRE probe includes a sharp tip. This tip drills the path to the treatment position. Typically, an IRE probe has to be rigid to allow piercing of the tissue but not too thick in order to reduce complications when advancing the probe, for example due to bleeding, and to ensure easy advancing. Thus, it is typically not possible to provide a flexible IRE probe that curves or follow a curved path to bypass a critical area. Injury to a critical area, such as an organ or blood vessel, by the IRE probe can result in an increased risk of bleeding or undesirable tissue damage from the intervention.

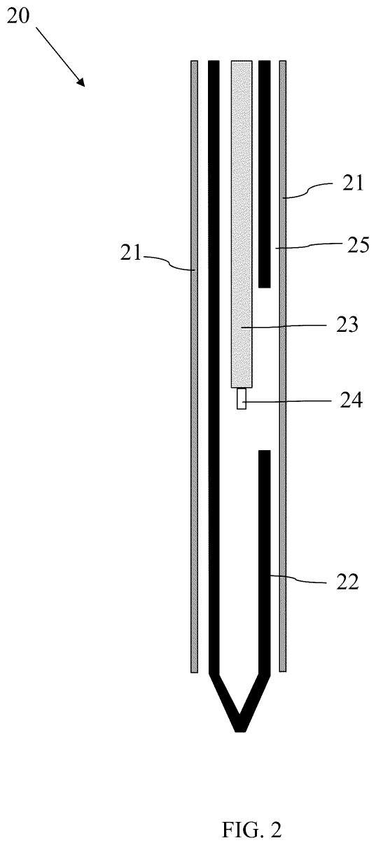

FIG. 2 shows an embodiment of an IRE probe system 20 of the present disclosure.

The IRE probe system 20 can include a sheath 21 having a lumen 25, a first electrode 22, a telescopic tube 23, and a second electrode 24. As shown, the first electrode 22 and the telescopic tube 23 with the second electrode 24 can be located inside the lumen 25. The sheath 21 can be retractable, steerable, or deflectable.

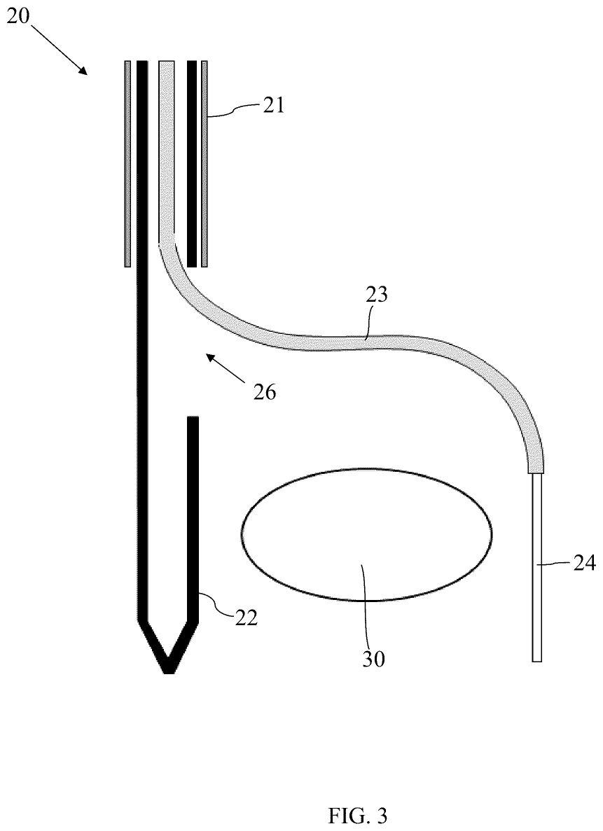

In use, the IRE probe system 20 can be inserted into tissue and positioned at one side of a target region 30, as shown in FIG. 3. The IRE probe system 20 can follow a vascular channel or be steered using imaging or electric field measurements to fine tune the placement. The sheath 21 can be retracted to expose the first electrode 22. The telescopic tube 23 can be bendable or a twice deflectable sheath that is pre-bent and extendable. FIG. 3 shows that the telescopic tube 23 can be first extended through an opening 26 in a direction perpendicular to a direction in which the first electrode 22 extends and continue to extend into a pre-bent “S” form including two opposing 90-degree bends. The second electrode 24 can then be extended from the telescopic tube 23 such that the first electrode 22 and the second electrode 24 are automatically positioned parallel to each other about the target region 30.

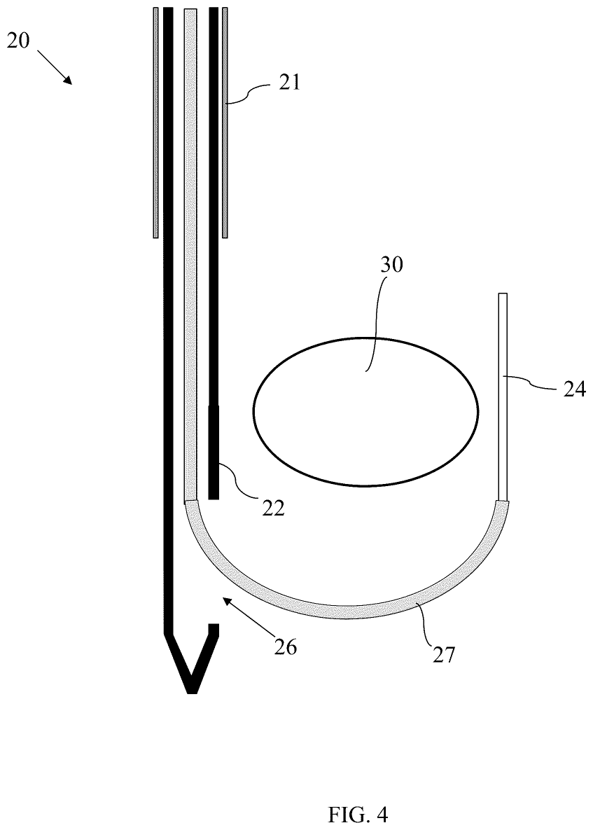

The telescopic tube 23 can be chosen from a kit to fit the specific needs for the treatment. Based on pre-operative planning (e.g. via simulation) a desired path of the second electrode 24 with respect to the size of the target region and electric field strength could be determined. The telescopic tube could be chosen as a result of this pre-operative planning in order to suit the treatment. Such a kit can include telescopic tubes 23 that are bendable or pre-bent with different dimensions or shapes. For example, in another aspect of the present invention, the telescopic tube can be pre-bent in a “U” shape, as shown in FIG. 4.

FIG. 4 shows an IRE probe system 20 similar to that shown in FIGS. 2 and 3. Descriptions of the same features as those described with respect to FIGS. 2 and 3 are omitted for brevity. However, the IRE probe system 20 shown in FIG. 4 includes a telescopic tube 27 that is pre-bent in a “U’ shape including a 180-degree bend.

FIG. 4 shows that the IRE probe system 20 can be inserted into tissue and positioned at one side of a target region 30. The sheath 21 can be retracted to expose the first electrode 22. The telescopic tube 27 can be first extended though the opening 26 in a direction perpendicular to a direction in which the first electrode 22 extends and continue to extend into its pre-bent “U” form under the target region 30. The second electrode 24 can then be extended from the telescopic tube 27 such that the first electrode 22 and the second electrode 24 are automatically positioned parallel to each other about the target region 30.

In another embodiment, rather than being pre-bent, as discussed with respect to FIGS. 3 and 4, a telescopic tube with an electrode at the end can be steerable. That is, the telescopic tube can be bendable and manipulated by a control device or mechanism that can be driven by (i) a medical professional assisted by imaging-guidance or (ii) a robot using control actuators.

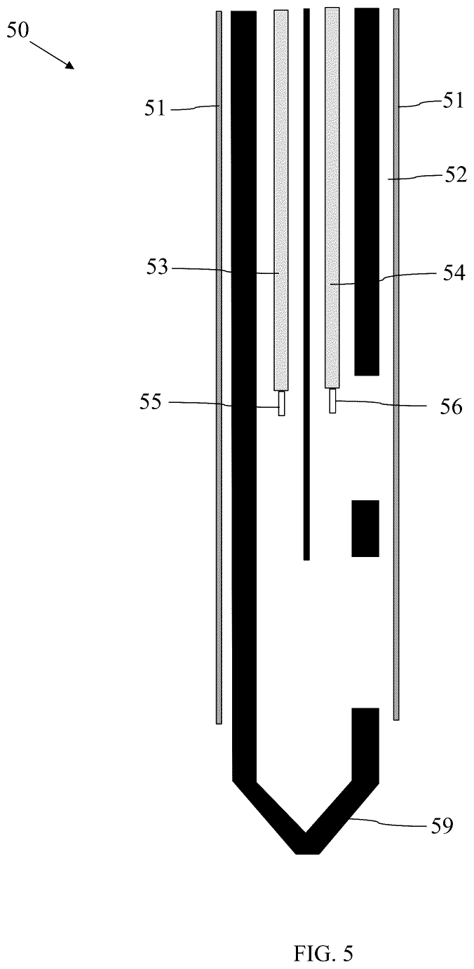

FIG. 5 shows another embodiment of an IRE probe system 50 of the present disclosure that includes two telescopic tubes with electrodes. The IRE probe system 50 can include a sheath 51 having a lumen 52, a tip 59, a first telescopic tube 53, a second telescopic tube 54, a first electrode 55, and a second electrode 24. As shown, the first telescopic tube 53 and the second telescopic tube 54 can be located inside the lumen 52. The sheath 51 can be retractable, steerable, or deflectable.

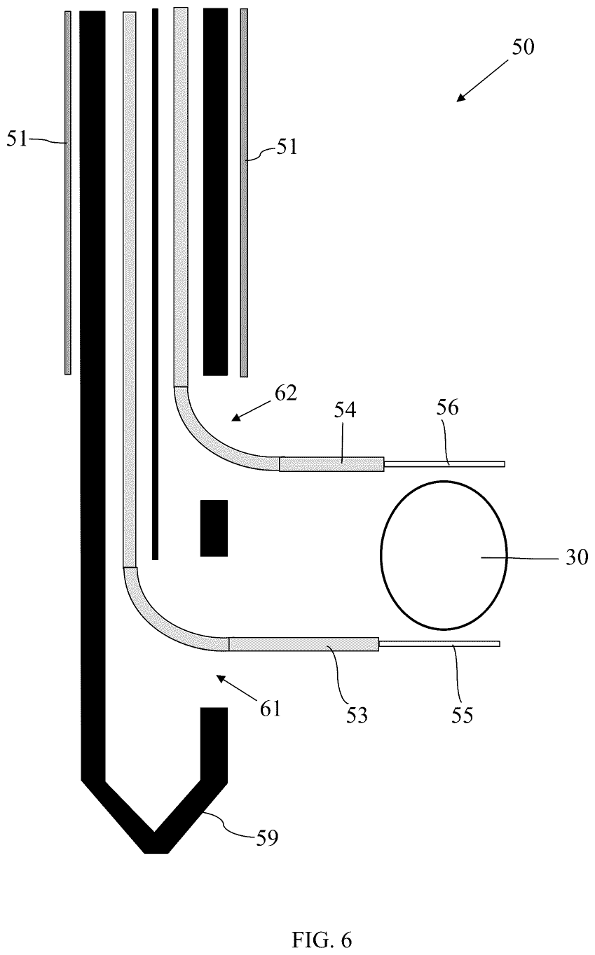

In use, the IRE probe system 50 can be inserted into tissue and positioned at one side of a target region 30, as shown in FIG. 6. The IRE probe system 50 can follow a vascular channel or be steered using imaging or electric field measurements to fine tune the placement. The sheath 51 can be retracted to expose first and second openings 61 and 62, respectively. The telescopic tubes 53 and 54 can be bendable or a once deflectable sheath that are pre-bent and extendable. FIG. 6 shows that the first telescopic tube 53 can be extended though the first opening 61 with a 90-degree bend in a direction perpendicular to a direction in which the tip 59 is extending. Likewise, the second telescopic tube 54 can be extended though the second opening 62 with a 90-degree bend in a direction perpendicular to a direction in which the tip 59 is extending. The first and second electrodes 55 and 56 can then be extended from their respective telescopic tubes 53, 54 such that the first electrode 55 and the second electrode 56 are automatically positioned parallel to each other about the target region 30.

While 90-degree and 180-degree bends in the telescopic tubes shapes are shown, the telescopic tubes can have various other shapes or contours as might be desired for a particular clinical application or treatment. The telescopic tubes can have any suitable shapes so long as the two electrodes parallel to one another.

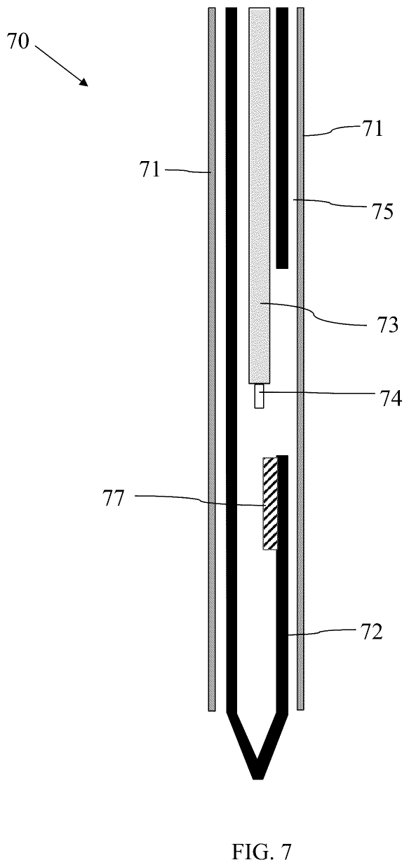

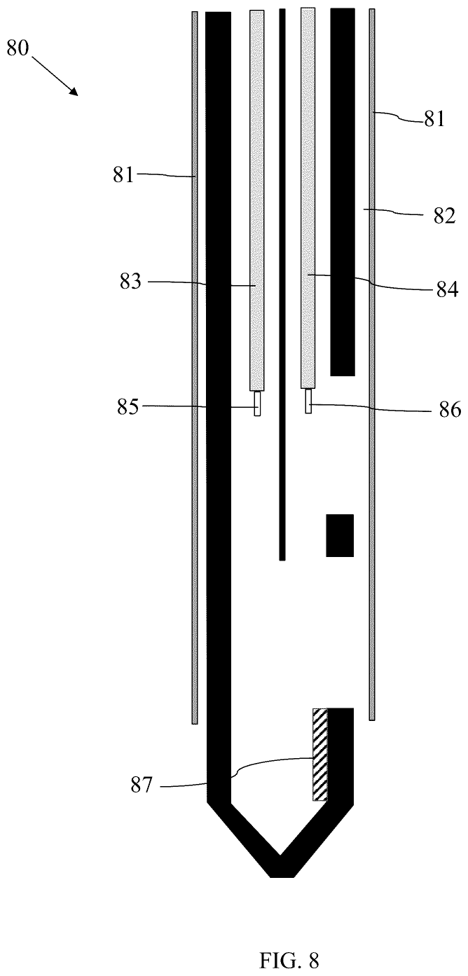

In other embodiments, an IRE probe system can include an imaging sensor in addition to ablation electrodes, as shown for example in FIGS. 7 and 8. Such an IRE probe system can permit multiple functions to be performed during an IRE ablation treatment such as both ablation and imaging.

For example, FIG. 7 shows an IRE probe system 70 that is similar to the IRE probe system 20 shown in FIG. 2. As shown in FIG. 7, the IRE probe system 70 can include a sheath 71 having a lumen 75, a first electrode 72, a telescopic tube 73, and a second electrode 74. However, the IRE probe system 70 can also include an imaging sensor 77.

In another example, FIG. 8 shows an IRE probe system 80 that is similar to the IRE probe system 50 shown in FIG. 5. As shown in FIG. 8, the IRE probe system 80 can include a sheath 81 having a lumen 82, a first telescopic tube 83, a second telescopic tube 84, a first electrode 85, and a second electrode 86. The IRE probe system 80 can also include an imaging sensor 87.

The imaging sensors 77 and 87 can be an ultrasound transducer, for example. In various examples, the imaging sensors 77, 87 can be an electronic ultrasound transducer, an all-optical ultrasound transducer, a piezoelectric transducers (PZT), capacitive micro-machined ultrasonic transducer (CMUT), a silicon photonics based ultrasound transducer, or a piezoelectric polyvinylidene fluoride (PVDF) based transducer. In other examples, other imaging sensors can be used.

When the IRE probe systems 70, 80 are positioned for ablation, the respective imaging sensors 77, 87 are positioned adjacent to the target region. This arrangement permits both ablation and imaging to be performed with one IRE probe system. In other examples, the IRE probe systems 70, 80 can be used with other sensors such as an external ultrasound transducer or another sensor arranged adjacent to the target region. In other examples (not shown), the IRE probe systems 70, 80 can include multiple sensors. Multiple imaging sensors can provide improved coverage of the target region and a more accurate representation of the tissue damaged during ablation compared to a conventional procedure using only a single imaging sensor.

In some examples, it may not be possible or desirable to perform imaging and ablation simultaneously as the electric field generated between IRE probe system electrodes can interfere with nearby imaging sensors. In such a case, ablation and imaging can be alternated. In other examples, such as when imaging sensors are configured as all-optical ultrasound sensors, the imaging and ablation functions can be performed simultaneously due to the lack of interference between the electrical field energy and the all-optical ultrasound sensor operation. The imaging sensors 77, 87 in this example, can be configured as an all-optical ultrasound transducer that allows simultaneous ablation and ultrasound imaging. All-optical transducers use pulsed or modulated light to generate ultrasound via the photoacoustic effect and can be less sensitive to electromagnetic noise than non-optical transducers that convert electrical energy to ultrasonic energy.

In some embodiments, the imaging sensors 77, 87 can be rigidly fixed to the respective IRE probe systems 70, 80. With this integration, the imaging sensors 77, 87 and the IRE probe electrodes are automatically physically registered with respect to each other, providing direct overlap of the ablation volume on the imaged ablation volume with minimal mismatch. The integration of the imaging sensors 77, 87 to the IRE probe systems 70, 80 does not require another imaging sensor to be separately positioned relative to the ablation volume. Because the imaging sensors 77, 87 are simultaneously and automatically located in the immediate vicinity of the ablation volume when the IRE probe systems 70, 80 are positioned at the target region 30, no special requirements to the power and sensitivity of the imaging sensors 77, 87 are needed. The power, sensitivity, and/or other operating parameters of the imaging sensors 77, 87 can be predetermined and easily configured. Accordingly, the IRE probe systems 70 and 80 provide various improvements over existing systems and devices. The IRE probe systems 70 and 80, for example, can permit simultaneous sensing/imaging and ablation. The integration of the imaging function into the IRE probe systems 70, 80 also provides for a cost-efficient implementation, and reduces the complexity of the IRE ablation treatment. Still further, the ablation treatment can be performed more effectively because of the improved imaging data that can be collected. This can improve the likelihood that the target tissue in the target region 30 is ablated during a single treatment and harm to surrounding and/or healthy tissues is minimized or reduced.

As mentioned, the IRE probe systems 70 and 80 can provide for simultaneous therapy and imaging. An all-optical ultrasound transducer can allow the electrodes and the imaging sensor to be operated simultaneously. Conventional piezoelectric transducers (PZT), capacitive micro-machined ultrasonic transducers (CMUT), and piezoelectric polyvinylidene fluoride (PVDF) based sensors cannot provide data to visualize the ablation area during the therapy mode due to electromagnetic interferences. The integration of the imaging sensors 77, 87 as an all-optical ultrasound transducer and/or as a silicon photonics based ultrasound transducer in the IRE probe systems 70, 80 can provide improved visualization of the ablation area over traditional or conventional treatments. The all-optical ultrasound transducers and/or the silicon photonics based ultrasound transducers can allow electromagnetic interference free detection of ultrasound during an ablation treatment.

The imaging sensors 77, 87 can be located within the respective lumens 72, 82 and be coupled to an imaging generator via a connector. In some examples, if the imaging sensors 77 or 87 are an all-optical ultrasound transducer, the connector can be to an optical fiber. In other examples, if other imaging sensors technology is used, such as electronic ultrasound sensors, the imaging sensor 77, 87 can be connected to an imaging generator via cables routed through lumens 72, 82. In still further examples, the connectors and/or cable can be located and routed along the sheath 71, 81 in a separate conduit structure.

In any of the embodiments or configuration previously described, the telescopic tube(s) including electrodes can be retracted after a first IRE ablation procedure in a first target region volume, then the IRE probe system can be rotated by a pre-defined angle and the telescopic tube(s) and electrodes can be extended into the tissue again to perform a second ablation in a second orientation of the target region volume. This can be repeated as necessary to treat the target region. This way, the target region coverage of an IRE probe system can be greatly expanded without having to perform other skin incisions or extraction and reinsertion of the same IRE probes or use multiple IRE probes. This is less invasive, more timely, and more efficient than convention IRE ablation procedures.

In any of the embodiments or configurations previously described, an IRE probe system can be moved or advanced manually or with a robotic system. In particular, the robotic system can specify a speed and direction for the movement of the IRE probe system toward a treatment position. If the IRE probe system includes a bendable tip, the robotic system can be designed to adapt the curvature of the tip in such a way or to deform the IRE probe system in such a way that the alignment of the tip through the curvature to the treatment position is adjusted. In other words, the tip of the IRE probe can be advanced with the robotic system in such a way that it is aligned in the direction of the treatment position. Thus, the direction of movement of the IRE probe can be predetermined at least in part by the robotic system.

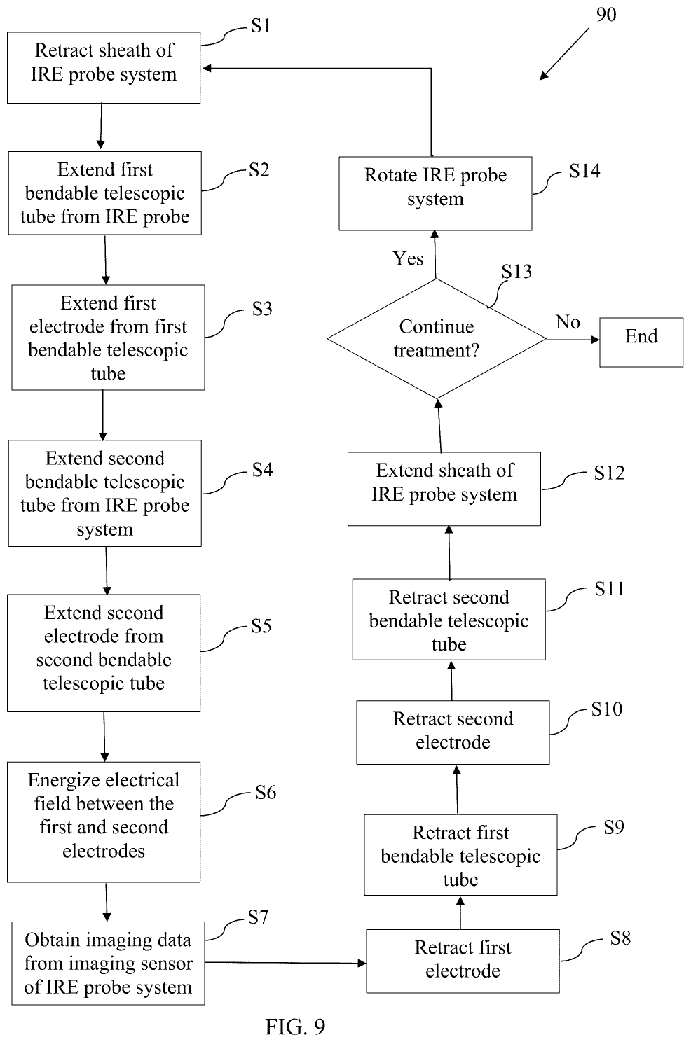

An example method 90 of performing an IRE ablation therapy is shown in FIG. 9. The example method 90 can be performed using one of the IRE probe systems previously described. While one or more example IRE probe systems are used to describe the method 90 below, it should be appreciated that the method 90 is not limited to specific examples detailed below and that other alternate embodiments and examples of IRE probe systems of the present disclosure can also be used. The order of steps as described can be changed and some steps omitted as optional depending on the IRE probe system used and treatment conditions.

The method 90 can begin at step S1 in which a sheath of a IRE probe system is retracted. While not shown, prior to the performance of step S1, the IRE probe system is positioned at or near a region of target tissue that requires IRE ablation therapy. The distal end of the IRE probe system can be inserted into a patient and to a treatment position adjacent to the target tissue, for example.

At step S2, a first bendable telescopic tube can be extended from a lumen of the IRE probe system. The first bendable telescopic tube can be as previously described and can be pre-bent to include a 90-degree bend, two opposing 90-degree bends, a 180-degree bend, or any other suitable shape or angle.

At step S3, a first electrode can be extended from the first bendable telescopic tube. This action can cause the first electrode to be oriented in parallel to a second electrode of the IRE probe system.

At step S4, a second bendable telescopic tube can be extended from a lumen of the IRE probe system. The second bendable telescopic tube can be as previously described and can be pre-bent to include a 90-degree bend or any other suitable shape or angle.

At step S5, a second electrode can be extended from the second bendable telescopic tube. This action can cause the second electrode to be oriented in parallel to the first electrode of the IRE probe system.

At step S6, an electrical field can be energized between the first electrode and the second electrode of the IRE probe system to ablate the target tissue in a first orientation between the first and the second electrodes. One of the first and second electrodes will be a positive electrode and the other of the first and second electrodes will be a negative electrode. The first and second electrodes can be energized with short high-voltage low-energy pulses to create an electrical field between the two electrodes to kill the cells (i.e., target tissue) in between the electrodes.

At step S7, imaging data can be obtained. The imaging data can be obtained from a first direction relative to the target tissue. The imaging data can be obtained from an imaging sensor located on the IRE probe system. The first imaging data can provide imaging information for one side or from one perspective of the target tissue.

The method 90 can be performed with an IRE probe system that includes an all-optical ultrasound transducer as the imaging sensor. In such case, the steps of S6 and S7 can be performed simultaneously. The all-optical ultrasound transducer can obtain imaging data concurrently while the first and second electrodes are energized to produce an electrical field.

At step S8, the first electrode can be retracted into the first bendable telescopic tube.

At step S9, the first bendable telescopic tube can be retracted into the lumen of the IRE probe system.

If a second electrode was extended from a second bendable telescopic tube, then the second electrode can also be retracted into the second bendable telescopic tube at step S10.

If a second bendable telescopic tube was extended from the lumen, then the second bendable telescopic tube can also be retracted into the lumen at step S11.

At step S12, the sheath of the IRE probe system can be extended. Extending the sheath can close any openings in the IRE probe systems to protect internal components and minimize features of the IRE probe system that can cause mechanical interference of surrounding tissue during extraction of the IRE probe system from the patient or during step S11.

At step S13, a decision is made to continue treatment or not. If the decision is to discontinue treatment, then the method ends and the IRE probe system can be extracted from the patient. If the decision is to continue treatment, then the method can continue to step S14.

At step S14, the IRE probe system can be rotated. The IRE probe system can be rotated a predefined amount. The degree of rotation can be defined during pre-treatment planning or as a result of imaging information obtained and processed by medical professionals during treatment.

After rotation in step S14, steps S1-S13 can be repeated to ablate target tissue from a second orientation. Rotating the IRE probe system and then extending electrodes from that new orientation will cause the first and second electrodes to have a different orientation relative to the target tissue volume. An electrical field energized between the two electrodes will also then affect the target tissue volume from a different direction. This method has an advantage that a greater target tissue volume can be treated without having to use multiple IRE probes or having to repeatedly extract and reinsert a IRE probe to different locations to treat the same target tissue volume.

Steps S1-S14 can be repeated as necessary to ablate the target tissue before ending the described method and extracting the IRE probe system from the patient.

It should be understood that the foregoing description is only illustrative of the present invention. Various alternatives and modifications can be devised by those skilled in the art without departing from the present invention. Accordingly, the present invention is intended to embrace all such alternatives, modifications, and variances that fall within the scope of the appended claims.

What is claimed is:

1. An ablation system comprising:
a retractable sheath including a lumen;
a first electrode in the lumen; and
a bendable telescopic tube extendable from the lumen and including a second electrode, wherein the first electrode and the second electrode are configured to deliver an electric field energy to target tissue in a patient.

2. The ablation system of claim 1, wherein the first electrode is exposed when the retractable sheath is retracted.

3. The ablation system of claim 1, wherein the bendable telescopic tube is pre-bent with two opposing 90-degree bends.

4. The ablation system of claim 1, wherein the bendable telescopic tube is pre-bent with a 180-degree bend.

5. The ablation system of claim 1, wherein the second electrode is extendable from the bendable telescopic tube.

6. The ablation system of claim 5, wherein when the bendable telescopic tube is extended from the lumen and the second electrode is extended from the bendable telescopic tube, the first electrode and the second electrode are arranged parallel to each other.

7. The ablation system of claim 1, further comprising an imaging sensor positioned to obtain imaging data of the target tissue.

8. The ablation system of claim 7, wherein the imaging sensor is configured to operate simultaneously with operation of the first and the second electrodes.

9. An ablation system comprising:
a retractable sheath including a lumen;
a first bendable telescopic tube extendable from the lumen and including a first electrode; and
a second bendable telescopic tube extendable from the lumen and including a second electrode, wherein the first electrode and the second electrode are configured to deliver an electric field energy to target tissue in a patient.

10. The ablation system of claim 9, wherein the first bendable telescopic tube and the second bendable telescopic tube are each pre-bent with a 90-degree bend.

11. The ablation system of claim 9, wherein
the first electrode is extendable from the first bendable telescopic tube, and
the second electrode is extendable from the second bendable telescopic tube.

12. The ablation system of claim 11, wherein when the first bendable telescopic tube is extended from the lumen, the first electrode is extended from the first bendable telescopic tube, the second bendable telescopic tube is extended from the lumen, and the second electrode is extended from the second bendable telescopic tube, the first electrode and the second electrode are arranged parallel to each other.

13. The ablation system of claim 9, further comprising an imaging sensor positioned to obtain imaging data of the target tissue.

14. The ablation system of claim 13, wherein the imaging sensor is configured to operate simultaneously with operation of the first and the second electrodes.

15. A method of performing irreversible electroporation (IRE) therapy comprising:
retracting a sheath of an IRE probe system that is located at or near target tissue in a patient;
extending a first bendable telescopic tube from a lumen of the IRE probe system;
extending a first electrode from the first bendable telescopic tube; and
energizing an electrical field between the first electrode and a second electrode of the IRE probe system that is parallel to the first electrode to ablate the target tissue in a first orientation.

16. The method of claim 15, further comprising:
retracting the first electrode;
retracting the first bendable telescopic tube;
extending the sheath;
rotating the IRE probe system;
re-extending the first bendable telescopic tube from the lumen of the IRE probe system;
re-extending the first electrode from the first bendable telescopic tube; and
energizing an electrical field between the first electrode and the second electrode to ablate the target tissue in a second orientation.

17. The method of claim 15, further comprising:
extending a second bendable telescopic tube from the lumen of the IRE probe system;
extending the second electrode from the second bendable telescopic tube.

18. The method of claim 17, further comprising:
retracting the first electrode and the second electrode;
retracting the first bendable telescopic tube and the second bendable telescopic tube;
extending the sheath;
rotating the IRE probe system;
re-extending the first bendable telescopic tube from the lumen of the IRE probe system;
re-extending the first electrode from the first bendable telescopic tube;
re-extending the second bendable telescopic tube from the lumen of the IRE probe system;
re-extending the second electrode from the second bendable telescopic tube; and
energizing an electrical field between the first electrode and the second electrode to ablate the target tissue in a second orientation.

19. The method of claim 15, further comprising obtaining imaging data from an imaging sensor of the IRE probe system.

20. The method of claim 19, further comprising simultaneously energizing the electrical field between the first electrode and the second electrode and obtaining imaging data from the imaging sensor.
